(19) United States
(12) Patent Application Publication
(10) Pub. No.: US 2023/0181279 A1
(43) Pub. Date: Jun. 15, 2023

(54) Cover For Sterile Field Fluid Systems

(71) Applicant: C Change Surgical, LLC, Winston-Salem, NC (US)

(72) Inventor: Patrick Kammer, Chapel Hill, NC (US)

(21) Appl. No.: 18/065,263

(22) Filed: Dec. 13, 2022

Related U.S. Application Data
(60) Provisional application No. 63/289,413, filed on Dec. 14, 2021.

(51) Int. Cl. A61B 50/00; A61B 46/10

(52) U.S. Cl. CPC A61B 50/00; A61B 46/10; A61B 2050/0051; A61B 2050/0066

(57) ABSTRACT

Covers for sterile field fluid systems can help maintain a sterile environment in a surgical field. A cover may include a top layer configured to be in contact with and/or connected to a bottom layer or a drape that may be positioned on an outer surface of the fluid system. The bottom layer may be configured to conform to a basin of the fluid system. The top layer may be sized and shaped to cover a cavity of the basin.

### CROSS-REFERENCE TO RELATED APPLICATIONS

This application claims priority to U.S. Provisional Pat. Application Serial No. 63/289,413 filed on Dec. 14, 2021, which is hereby incorporated by reference as though fully set forth herein.

### FIELD

Described herein are covers for sterile field fluid systems, for example, a cover for a surgical basin used to control the temperature of surgical materials. The covers can be useful for maintaining a sterile environment in a surgical field.

### BACKGROUND

Sterile field fluid systems, such as surgical slush machines, heated fluid basins, and the like, can be used during various medical procedures. For example, surgical slush machines can be to used rapidly cool tissue or organs during surgery and heated fluid basins may be used to maintain sterile fluid at or near body core temperature for irrigation during surgery. Sterile field fluid systems may be open to the atmosphere of the surgical field and become exposed to contaminants, which can increase the risk of complications during or subsequent to the various medical procedures.

### SUMMARY

The present disclosure is directed to covers for sterile field fluid systems. Conventional surgical drapes may be used to line a basin of a sterile field fluid system to provide a barrier between the basin and the fluid, however, these drapes allow for the sterile fluid and other contents of the basin to be exposed to the atmosphere of the surgical field and other contaminants that may enter the basin.

In some examples, a cover may include a bottom layer and a top layer. The bottom layer may be positioned on an outer surface of the fluid system. The top layer may be adjacent to the bottom layer. The top layer may have a surface area that is smaller than a surface area of the bottom layer. At least a portion of the top layer may be connected to the bottom layer. The bottom layer may be configured to conform to a basin of the fluid system. The top layer may be sized to cover a cavity of the basin.

In other examples, a cover may include a sterile top layer that may be positioned on a drape that covers a basin of a fluid system. The sterile top layer may have a surface area that is smaller than a surface area of the drape. At least a portion of the sterile top layer may be connected to the drape. The sterile top layer may be sized to cover a cavity of a basin of the fluid system.

In yet other examples, a method may be used for covering a surgical basin of a sterile field fluid system. A surgical basin of a sterile field fluid system may be covered with a bottom layer of a cover. The surgical basin may include sidewalls that define a cavity. The bottom layer of the cover may be positioned on an outer surface of the sterile field fluid system. The bottom layer may conform to the sidewalls of the surgical basin. Surgical material may be positioned in the cavity of the surgical basin. The cavity of the surgical basin may be covered with a top layer of the cover. The top layer may be adjacent to the bottom layer. The top layer may be at least partially connected to the bottom layer. The top layer may have a surface area that is smaller than a surface area of the bottom layer.

### BRIEF DESCRIPTION OF FIGURES

FIG. 1 is a perspective view of a conventional surgical basin.

FIG. 2 is a perspective view of a conventional sterile field fluid system that includes a conventional surgical basin.

FIG. 3 is a sectional side-view of a cover with a surgical basin according to one example of the present disclosure.

FIG. 4 is a top-view of a cover with a surgical basin according to one example of the present disclosure.

FIG. 5 is a sectional side-view of a sterile field fluid system that includes a cover according to one example of the present disclosure.

FIG. 6 is a top-view of a top layer for a cover according to one example of the present disclosure.

### DETAILED DESCRIPTION

Provided herein are covers, for surgical basins, that include a top layer and a bottom layer. A surgical basin may include any suitable container for forming, storing, or otherwise accessing surgical material. Surgical material may include surgical fluids, such as surgical slush sterile saline slush, heated surgical fluid, organs, tissue, surgical tools or any other suitable material that may need to be stored and optionally temperature controlled during a surgical procedure. The surgical basin may include sidewalls that define a cavity of the surgical basin. The cavity of the basin may have a depth and width sized to hold the necessary surgical material for a medical procedure. The bottom layer of the cover may be configured to conform to the surface of surgical basin to the full depth of the cavity. The top layer may be sized to cover the opening of the cavity of the surgical basin and any contents thereof.

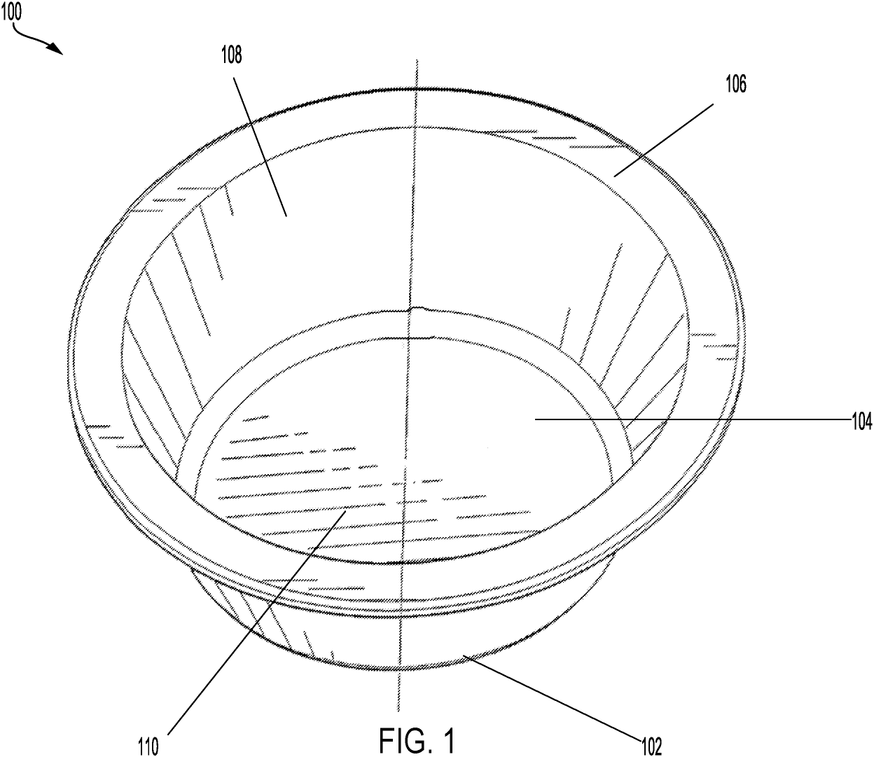

FIG. 1 is a perspective view of a conventional surgical basin 100. A surgical basin may include a body 102 that includes sidewalls 108, and bottom surface 110 to form a cavity 104, and a perimeter 106. The body 102 may be shaped in various configurations to create a cavity 104 having a cross-sectional shape that may be trapezoidal, rectangular, elliptical, circular, kidney, or other shapes useful for holding or otherwise retaining surgical material. The perimeter 106 of basin 100 may include any suitable shape for a perimeter of a surgical basin. For example, as illustrated in FIG. 1, the perimeter 106 may be circular, but the perimeter may alternatively be elliptical, square, rectangular, triangular, hexagonal, octagonal, kidney, or any other suitable shape. In some examples, a drape (not shown) may be positioned in the cavity 104 of surgical basin 100.

A surgical basin may comprise a metal, a metal alloy, a polymer composite, or any other suitable material for use to contain a surgical basin. The surgical material may include surgical slush, heated fluid, organs, tissue, or other suitable material that may be used during a medical procedure.

A surgical basin may be used during one or more medical procedures. For example, a surgical slush may rapidly cool tissue or organs such as a heart during a heart transplant to prevent or mitigate permanent damage to the tissue or organs. A heated fluid may be used for irrigation techniques or other suitable techniques during the medical procedure. The surgical slush, the heated fluid, or other suitable material for use during the medical procedure may be positioned in the cavity of the basin.

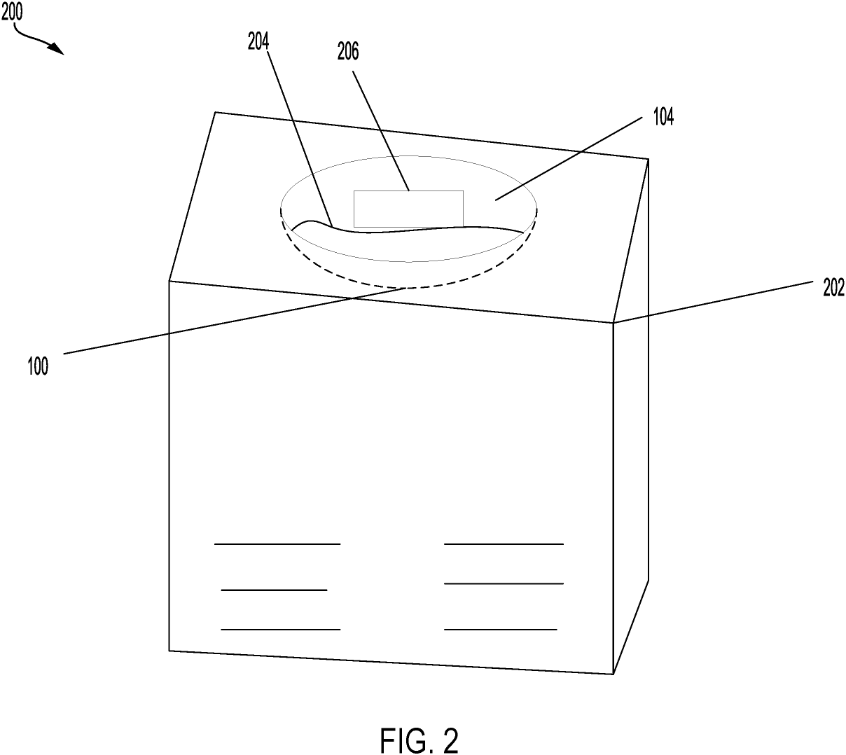

FIG. 2 is a perspective view of a conventional sterile field fluid system 200 that includes basin 100 and fluid preparation device 202. In some examples, fluid preparation device 202 may include a chilling device, a heating device, or other suitable device that may be used to generate sterile fluid for use in medical procedures. Surgical fluid 204 may be positioned within the cavity 104 of basin 100. Surgical material 206 may be positioned in cavity 104 of basin 100 with or without fluid 204. For example, surgical material 206, such as a liver, may be positioned in basin 100 with fluid 204 to prevent or mitigate damage to, or complications associated with, surgical material 206.

A chilling device may generate or otherwise prepare surgical slush that may rapidly cool tissue or organs to reduce a metabolic rate of the tissue or organs to prevent or mitigate damage. A heating device may heat fluid such as saline or other suitable solution to approximate body temperature for irrigation or other techniques.

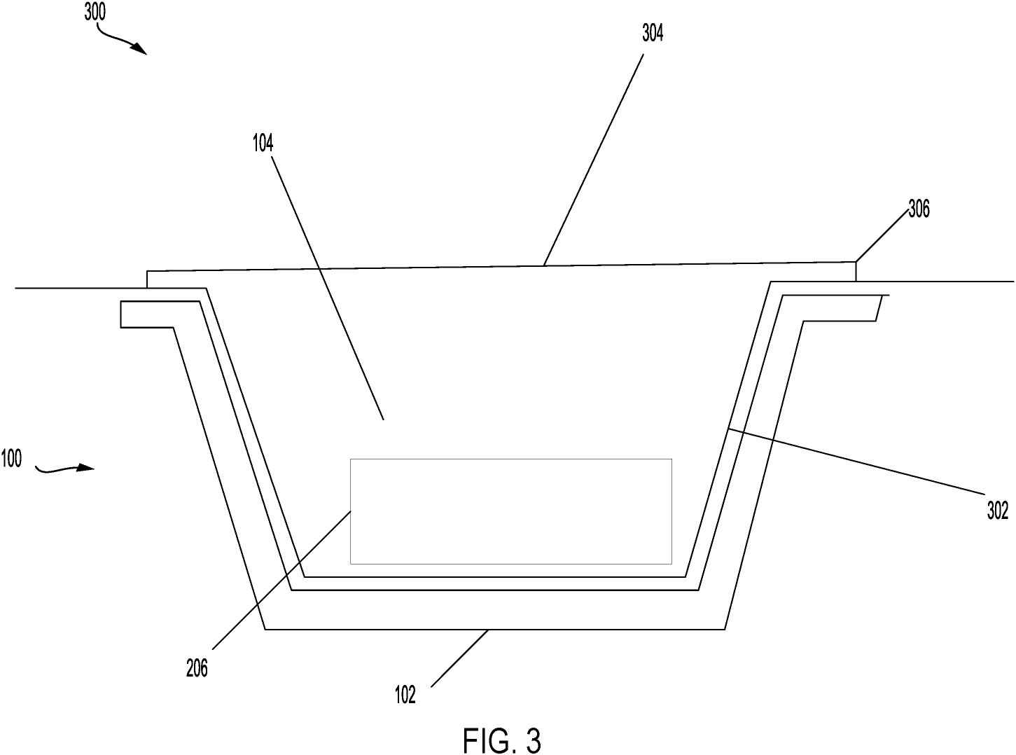

FIG. 3 is a sectional side-view of a cover 300 that may be used with a basin 100 according to one example of the present disclosure. The cover 300 may include a bottom layer 302 and a top layer 304. Optionally, the top layer 304 and the bottom layer 302 may be integrally formed. Bottom layer 302 may contact and line the surface of basin 100, prior to placement of surgical material 206. Top layer 304 may be placed above basin 100 and bottom layer 302 to provide a barrier to the environment for surgical material 206. In some examples, cover 300 may include other suitable component or combination of components.

In some examples, a cover may comprise a textile, polymer, or other any suitable material for use with a sterile field fluid system. A cover, or any portion thereof, may comprise a flexible material that can conform to the surface of the basin and cover the sterile field fluid system. A cover, or any portion thereof, may comprise a material suitable to be molded to conform to the surface of the basin and cover the sterile field fluid system. A cover as described herein may comprise a material that may be pre-sterilized or suitable to be sterilized and/or re-sterilized. In some examples, a cover may be disposable (e.g., a single-use item), re-sterilized and reused, or recyclable.

In some examples, the top layer , the bottom layer, or both may include one or more ply or internal layers. For example, the top layer, the bottom layer, or a combination thereof may include one-ply, two-ply, three-ply, or any other suitable amount of internal layers. In some examples, the ply or internal layers of the top layer, the bottom layer, or a combination thereof may be sterilized or sterilizable.

In some examples, the top layer may be adjacent to the bottom layer, and at least a portion of the top layer may be connected to the bottom layer. For example, a portion of perimeter 306 or the entire perimeter 306 of top layer 304 may be connected to bottom layer 302. The top layer 304 may be connected to bottom layer 302 via an adhesive bond or connection, a mechanical bond or connection, or any other suitable connection means. For example, the top layer may be connected to the bottom layer via an epoxy or other suitable chemical bond, a thermal or ultrasonic weld, an interlocking mechanism (e.g., corresponding ridges that, when aligned and adjacent, may form a seal), or other means known to those of skill in the art.

The bottom layer 302 may be sized or shaped to conform to basin 100. For example, as illustrated in FIG. 3, the bottom layer 302 follows the contours of the body 102 of basin 100. The top layer 304 may include a shape that is different, similar, or identical to a shape of (or otherwise included in basin 100. For example, a rectangular top layer may be used to cover a rectangular cavity in a basin. The top layer may be sized to cover the cavity of the basin. For example, a circular top layer may be used to cover a circular-shaped cavity, where the diameter of the top layer is greater than the diameter of the cavity and/or basin. The top layer be configured to be adjusted with respect to the bottom layer to provide full or partial access to the cavity. For example, the top layer 304 may be configured to be pivoted or otherwise adjusted to expose at least a portion of the basin.

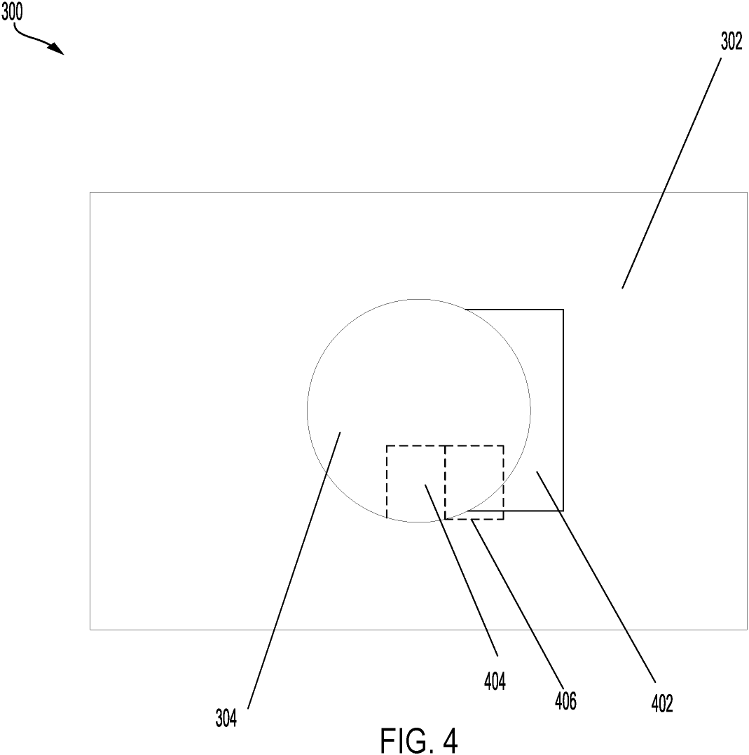

FIG. 4 is a top-view of cover 300. Cover 300 may include top layer 304, bottom layer 302, and optionally, tab 402. The cover 300 may include any other suitable components for covering a surgical basin for mitigating or preventing contamination of the surgical basin or materials within the basin. As illustrated in FIG. 4, the bottom layer 302 may include a first surface area, and the top layer 304 may include a second surface area. In some examples, the first surface area may be greater than the second surface area. Tab 402 may be affixed to top layer 304 and may allow the top layer 304 to be pivoted or otherwise adjusted, for example to provide at least partial access to the surgical basin 100. In some examples, tab 402 may be integrated into the shape or profile of top layer 304.

In some examples, the top layer 304 may include an opening 404. The top layer may include one opening, two openings, three openings, or any other suitable amount of openings. In other examples, the top layer may not include an opening and may, instead, be adjustable for providing access to the contents of the surgical basin. In some examples, opening 404 may be positioned to allow at least partial access to the cavity of a surgical basin. For example, the opening 404 may expose at least a portion of the surgical basin 100. Opening 404 may be positioned substantially in the center of the top layer 304 which may align with the center of the cavity. In other examples, opening 404 may be positioned off-center to align with a proximate edge of the cavity.

In some examples, the opening may comprise a slit, or other suitable openings or opening mechanisms. In some examples, the top layer 304 may optionally include a flap 406 that may be positioned adjacent to the opening 404 and be configured to substantially seal opening 404. In some examples, the flap may comprise a repositionable adhesive, a mechanical closure, other suitable sealing mechanisms, or a combination thereof. The repositionable adhesive, the mechanical closure, or a combination thereof may be configured to be reused, for example, a sufficient amount of times for facilitating one or more medical procedures.

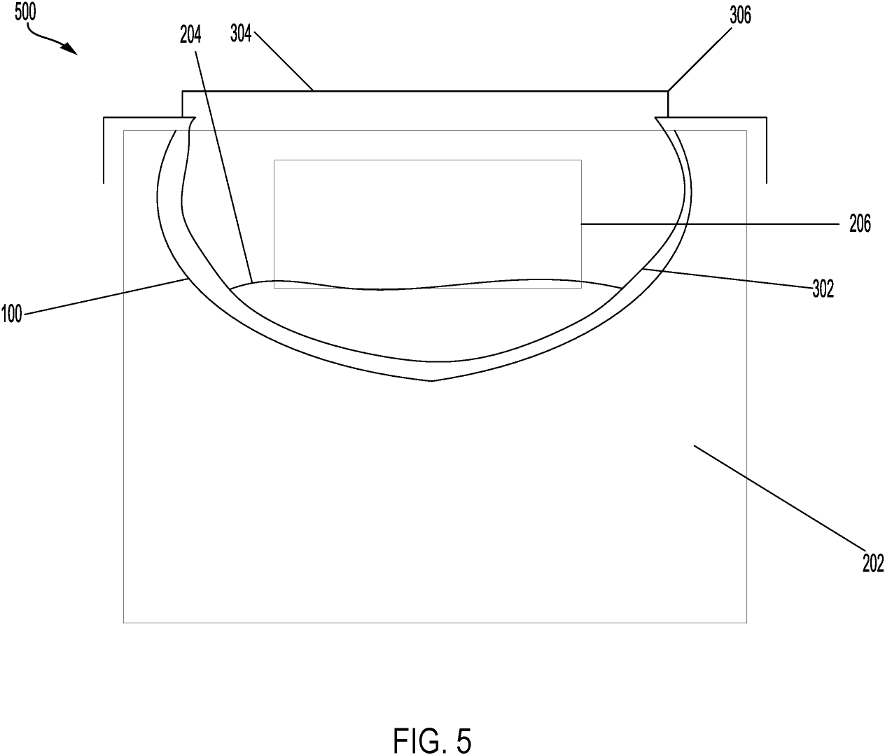

FIG. 5 is a sectional side-view of a sterile field fluid system 500 with cover 300. The sterile field fluid system 500 may include basin 100 and device 202. The surgical basin 100 may be positioned in the fluid preparation device 202, which may heat, chill, or perform other suitable functions with respect to basin 100, which may include surgical fluid 204 and/or surgical material 206. In some examples, the cover 300 (e.g., via the top layer 304) may be adjustable for allowing temporary and partial access to the material of the surgical basin.

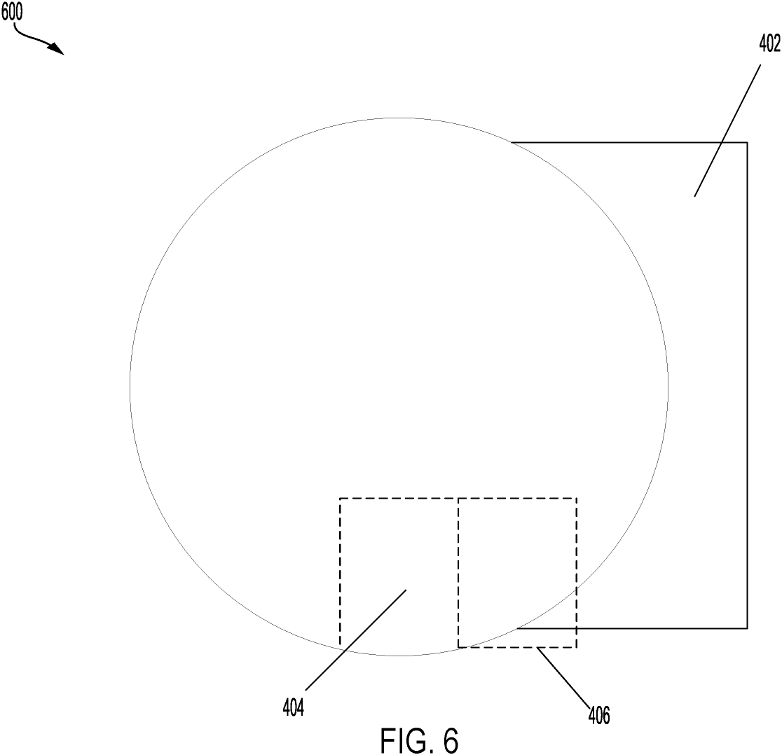

FIG. 6 is a top-view of a top layer 600. The top layer 600 may include tab 402, opening 404 (or more than one opening 404), flap 406, any other suitable components for the top layer 600, or any suitable combination thereof. The top layer 600 may include various suitable materials. For example, the top layer 600 may include a flexible plastic material, a rigid polymeric material, or other suitable materials that may be sterilized (e.g., for single-use or for reuse or re-sterilization). In some examples, the top layer 600 may be similar or identical to the top layer 304 of the cover 300 except that the top layer 600 may be formed or otherwise produced separately from the cover 300. The top layer 600 may be connected to the bottom layer 302 of the cover 300 during (e.g., while preparing for) a medical procedure and may be removed from the cover 300 subsequent to the medical procedure.

In some examples, the top layer may be a sterile layer that may be positioned on, or otherwise connected to, a drape that may be used with a surgical basin. A drape may cover the surgical basin, and at least a portion of the top layer may be in contact with the drape for covering a cavity of a basin that may comprise surgical material. In some examples, the cover may comprise a rigid material with sufficient mass to maintain the position of the cover when placed on the draped basin. The cover may be substantially free of adhesive and provide for ease of removal from a draped basin as needed during a procedure. In some examples, at least a portion of the top layer may be affixed or otherwise connected to the drape covering a cavity of a basin that may comprise surgical material. The top layer may be connected via a perimeter of the top layer to a drape via an adhesive connection, a mechanical connection, or a combination thereof. For example, the top layer may be connected to the drape via an epoxy or other suitable chemical bond, a thermal or ultrasonic weld, an interlocking mechanism (e.g., corresponding ridges that, when aligned and adjacent, may form a seal), or other means known to those of skill in the art.

The top layer may include a surface area that is smaller than a surface area of the drape. The top layer may include a shape that may be substantially the same as, or otherwise similar to, a shape of the cavity of the basin. For example, the shape of the top layer may be circular, elliptical, square, rectangular, hexagonal, octagonal, triangular, kidney, or any other suitable shape of, or corresponding to, the cavity and basin. In FIG. 6, opening 404 of the top layer 600 is positioned near a perimeter 602 of the top layer 600. In some examples, opening 404 may be positioned in any other suitable location (e.g., centrally located, off-center, at an edge, and the like) with respect to the top layer 600, the drape, or the surgical basin.

The opening 404 of the top layer 600 may allow at least partial access to the cavity of the surgical basin. The opening 404 may include a slit other suitable openings, or any suitable combination thereof. Optionally, a flap 406 may be positioned adjacent or otherwise proximate to the opening 404. The flap 406 may substantially seal the opening 404. The flap 406 may include a repositionable adhesive or a mechanical closure. The repositionable adhesive or the mechanical closure may allow the flap 406 to be repositioned multiple times and allow the flap 406 to substantially seal the opening 404 subsequent to being repositioned multiple times.

### Illustrative Embodiments of Suitable Compositions and Methods

As used below, any reference to methods, products, or systems is understood as a reference to each of those methods, products, or systems disjunctively (e.g., “Illustrative embodiment 1-4 is understood as illustrative embodiment 1, 2, 3, or 4.”).

Illustrative embodiment 1 is a cover for a sterile field fluid system, the cover comprising: a bottom layer positionable on an outer surface of the fluid system; and a top layer adjacent to the bottom layer, the top layer having a surface area smaller than a surface area of the bottom layer, wherein at least a portion of the top layer is connected to the bottom layer, wherein the fluid system comprises a basin having sidewalls defining a cavity, wherein the bottom layer is configured to conform to the basin and the top layer is sized to cover the cavity of the basin.

Illustrative embodiment 2 is the cover of any of the preceding or subsequent embodiments, wherein the top layer is connected to the bottom layer by an adhesive, bond, thermal weld, ultrasonic weld, interlocking mechanism, or combinations thereof.

Illustrative embodiment 3 is the cover of any of the preceding or subsequent embodiments, wherein a shape of the top layer is substantially the same as a shape of the cavity.

Illustrative embodiment 4 is the cover of any of the preceding or subsequent embodiments, wherein the perimeter of the top layer is connected to the bottom layer.

Illustrative embodiment 5 is the cover of any of the preceding or subsequent embodiments, wherein the surface of the top layer defines at least one opening that allows access to the cavity.

Illustrative embodiment 6 is the cover of any of the preceding or subsequent embodiments, wherein the at least one opening comprises a slit.

Illustrative embodiment 7 is the cover of any of the preceding or subsequent embodiments, wherein the top layer comprises a flap adjacent to the at one opening configured to substantially seal the at least one opening.

Illustrative embodiment 8 is the cover of any of the preceding or subsequent embodiments, wherein the flap comprises a repositionable adhesive or mechanical closure.

Illustrative embodiment 9 is the cover of any of the preceding or subsequent embodiments, wherein the at least one opening is positioned substantially over the center of the cavity.

Illustrative embodiment 10 is the cover of any of the preceding or subsequent embodiments, wherein the top layer is pivotable away from the bottom layer to provide access to the cavity.

Illustrative embodiment 11 is the cover of any of the preceding or subsequent embodiments, wherein the top layer comprises a material suitable for reuse or re-sterilization.

Illustrative embodiment 12 is the cover of any of the preceding or subsequent embodiments, wherein the top layer, the bottom layer, or both comprise a plurality of ply.

Illustrative embodiment 13 is the cover of any of the preceding embodiments, wherein the top layer, the bottom layer, or both comprise a pre-sterilized material.

Illustrative embodiment 14 is a cover for a sterile field fluid system, the cover comprising: a top layer positionable on a drape covering a basin of a fluid system, the top layer having a surface area smaller than a surface area of the drape, wherein at least a portion of the top layer is in contact with the drape, wherein the basin comprises sidewalls defining a cavity, and wherein the top layer is sized to cover the cavity of the basin.

Illustrative embodiment 15 is the cover of any of the preceding or subsequent embodiments, wherein the top layer comprises an adhesive, interlocking mechanism, or combinations thereof to connect the top layer to the drape.

Illustrative embodiment 16 is the cover of any of the preceding or subsequent embodiments, wherein a shape of the top layer is substantially the same as a shape of the cavity.

Illustrative embodiment 17 is the cover of any of the preceding or subsequent embodiments, wherein the perimeter of the top layer is in contact with the drape.

Illustrative embodiment 18 is the cover of any of the preceding or subsequent embodiments, wherein the surface of the top layer defines at least one opening that allows access to the cavity.

Illustrative embodiment 19 is the cover of any of the preceding or subsequent embodiments, wherein the at least one opening comprises a slit.

Illustrative embodiment 20 is the cover of any of the preceding or subsequent embodiments, wherein the top layer comprises a flap adjacent to the at one opening configured to substantially seal the at least one opening.

Illustrative embodiment 21 is the cover of any of the preceding or subsequent embodiments, wherein the flap comprises a repositionable adhesive or mechanical closure.

Illustrative embodiment 22 is the cover of any of the preceding or subsequent embodiments, wherein the at least one opening is positioned substantially over the center of the cavity.

Illustrative embodiment 23 is the cover of any of the preceding or subsequent embodiments, wherein the top layer is pivotable away from the drape to provide access to the cavity.

Illustrative embodiment 24 is the cover of any of the preceding or subsequent embodiments, wherein the top layer comprises a material suitable for reuse or re-sterilization.

Illustrative embodiment 25 is the cover of any of the preceding or subsequent embodiments, wherein the top layer comprises a plurality of ply.

Illustrative embodiment 26 is the cover of any of the preceding embodiments, wherein the top layer comprises a pre-sterilized material.

Illustrative embodiment 27 is a method comprising: covering a surgical basin of a sterile field fluid system with a bottom layer of a cover, the surgical basin including sidewalls defining a cavity, the bottom layer of the cover positioned on an outer surface of the sterile field fluid system and configured to conform to the sidewalls of the surgical basin; positioning surgical material in the cavity of the surgical basin; and covering the cavity of the surgical basin with a top layer of a cover, the top layer adjacent to the bottom layer and at least partially connected to the bottom layer, the top layer having a surface area smaller than a surface area of the bottom layer.

Illustrative embodiment 28 is a method comprising: covering a surgical basin of a sterile field fluid system with a cover of any of illustrative embodiments 1-26.

Whereas various examples of the disclosure have been described in fulfillment of the various objectives of the disclosure, it should be recognized that these examples are merely illustrative of the principles of the present disclosure. Numerous modifications and adaptations thereof will be readily apparent to those skilled in the art without departing from the spirit and scope of the present disclosure as defined in the following claims.

As used herein, unless otherwise expressly specified, all numbers such as those expressing values, ranges, amounts or percentages may be read as if prefaced by the word “about”, even if the term does not expressly appear. Any numerical range recited herein is intended to include all subranges subsumed therein. Plural encompasses singular and vice versa. Including and like terms means “including but not limited to”. The word “comprising” and forms of the word “comprising” as used in this description and in the claims does not limit the disclosure to exclude any variants or additions. Although the disclosure has been described in terms of “comprising”, “consisting essentially of” or “consisting of” are also within the scope of the present disclosure.

What is claimed is:

1. A cover for a sterile field fluid system, the cover comprising:
a bottom layer positionable on an outer surface of the sterile field fluid system; and
a top layer adjacent to the bottom layer, the top layer having a surface area smaller than a surface area of the bottom layer,
wherein at least a portion of the top layer is connected to the bottom layer,
wherein the sterile field fluid system comprises a basin having sidewalls defining a cavity, and
wherein the bottom layer is configured to conform to the basin and the top layer is sized to cover the cavity of the basin.

2. The cover of claim 1, wherein the top layer is connected to the bottom layer by an adhesive, bond, thermal weld, or ultrasonic weld, interlocking mechanism, or combinations thereof.

3. The cover of claim 1, wherein a shape of the top layer is substantially the same as a shape of the cavity.

4. The cover of claim 1, wherein a perimeter of the top layer is connected to the bottom layer.

5. The cover of claim 1, wherein a surface of the top layer defines at least one opening that allows access to the cavity.

6. The cover of claim 5, wherein the at least one opening comprises a slit.

7. The cover of claim 5, wherein the top layer comprises a flap adjacent to the at least one opening configured to substantially seal the at least one opening.

8. The cover of claim 7, wherein the flap comprises a repositionable adhesive or mechanical closure.

9. The cover of claim 5, wherein the at least one opening is positioned substantially over the center of the cavity.

10. The cover of claim 1, wherein the top layer is pivotable away from the bottom layer to provide access to the cavity.

11. The cover of claim 1, wherein the top layer comprises a material suitable for reuse or re-sterilization.

12. The cover of claim 1, wherein the top layer, the bottom layer, or both comprise a plurality of ply.

13. The cover of claim 1, wherein the top layer, the bottom layer, or both comprise a pre-sterilized material.

14. A cover for a sterile field fluid system, the cover comprising:
a top layer positionable on a drape covering a basin of a fluid system, the top layer having a surface area smaller than a surface area of the drape,
wherein at least a portion of the top layer is in contact with the drape,
wherein the basin comprises sidewalls defining a cavity, and
wherein the top layer is sized to cover the cavity of the basin.

15. The cover of claim 14, wherein the top layer comprises an adhesive, interlocking mechanism, or combinations thereof to connect the top layer to the drape.

16. The cover of claim 14, wherein a perimeter of the top layer is in contact with the drape.

17. The cover of claim 14, wherein a surface of the top layer defines at least one opening that allows access to the cavity.

18. The cover of claim 17, wherein the top layer comprises a flap adjacent to the at least one opening configured to substantially seal the at least one opening.

19. The cover of claim 14, wherein the top layer is pivotable away from the drape to provide access to the cavity.

20. A method comprising:
covering a surgical basin of a sterile field fluid system with a bottom layer of a cover, the surgical basin including sidewalls defining a cavity, the bottom layer of the cover positioned on an outer surface of the sterile field fluid system and configured to conform to the sidewalls of the surgical basin;
positioning surgical material in the cavity of the surgical basin; and
covering the cavity of the surgical basin with a top layer of a cover, the top layer adjacent to and at least partially connected to the bottom layer, the top layer having a surface area smaller than a surface area of the bottom layer.
